SEQUENCE LISTING (35 sequences)

SEQ ID NO: 1 - DNA, 18 bp, synthetic construct
  source 1..18: mol_type = other DNA
gtctacatgttttggtcc

SEQ ID NO: 2 - DNA, 19 bp, synthetic construct
  source 1..19: mol_type = other DNA
tgtctacatgttttggtcc

SEQ ID NO: 3 - DNA, 108 bp, synthetic construct
  source 1..108: mol_type = other DNA
cgcccgcagcccacctctcgggggcgggctcccggcgctagcagggctgaagagaagatggaggagctggtggtggaagtgcggggctccaatggcgctttctacaag

SEQ ID NO: 4 - DNA, 1724 bp, synthetic construct
  source 1..1724: mol_type = other DNA
cattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgccatgaaacttcaggtaattaactgagagtatattattactagggcattttttttttaactgagcgaaaatatttttgtgcccctaagaacttgaccacatttcctttgaatttgtggtgttgcagtggactgaattgttgaggctttatataggcattcatgggtttactgtgctttttaaagttacaccattgcagatcaactaacacctttcagttttaaaaggaagatttacaaatttgatgtagcagtagtgcgtttgttggtatgtaggtgctgtataaattcatctataaattctcatttccttttgaatgtctataacctctttcaataatatcccaccttactacagtattttggcaatagaaggtgcgtgtggaaggaaggctggaaaatagctattagcagtgtccaacacaattcttaaatgtattgtagaatggcttgaatgtttcagacaggacacgtttggctataggaaaataaacaattgactttattctgtgtttaccaattttatgaagacatttggagatcagtatatttcataaatgagtaaagtatgtaaactgttccatactttgagcacaaagataaagccttttgctgtaaaaggaggcaaaaggtaaccccgcgtttatgttcttaacagtctcatgaatatgaaattgtttcagttgactctgcagtcaaaattttaatttcattgattttattgatccataatttcttctggtgagtttgcgtagaatcgttcacggtcctagattagtggttttggtcactagatttctggcactaataactataatacatatacatatatatgtgtgagtaacggctaatggttaggcaagattttgattgacctgtgatataaacttagattggatgccactaaagtttgcttatcacagagggcaagtagcacattatggccttgaagtacttattgttctcttccagcaacttatgatttgctccagtgattttgcttgcacactgactggaatataagaaatgccttctatttttgctattaattccctccttttttgttttgttttgtaacgaagttgtttaacttgaaggtgaatgaagaataggttggttgccccttagttccctgaggagaaatgttaatacttgaacaagtgtgtgtcagacaaattgctgttatgtttatttaattaagtttgatttctaagaaaatctcaaatggtctgcactgatggaagaacagtttctgtaacaaaaaagcttgaaatttttatatgacttataatactgctgtgagttttaaaagtaaagcaaaagtaaactgagttgcttgtccagtgggatggacaggaaagatgtgaaataaaaaccaatgaaaaatgaa

SEQ ID NO: 5 - DNA, 420 bp, synthetic construct
  source 1..420: mol_type = other DNA
agcggggggcgcttcagccgggccgcctcctgcagcgccaagagggcttcaggtctcctttggcttctcttttccggtctagcattgggacttcggagagctccactgttctgggcgagggctgtgaagaaagagtagtaagaagcggtagtcggcaccaaatcacaatggcaactgatttttagtggcttctctttgtggatttcggaggagattttagatccaaaagtttcaggaagaccctaacatggcccagcagtgcattgaagaagttgatcatcgtgaatattcgcgtccccctttttgttaaacggggtaaattcaggaatgcacatgcttcagcgtctaaaaccattagcagcgctgctacttaaaaattgtgtgtgtgtgtttaagtttccaaagacctaaatatatgcc

SEQ ID NO: 6 - DNA, 20 bp, synthetic construct
  source 1..20: mol_type = other DNA
agaagccaaaggagacctga

SEQ ID NO: 7 - DNA, 20 bp, synthetic construct
  source 1..20: mol_type = other DNA
aaagagaagccaaaggagac

SEQ ID NO: 8 - DNA, 22 bp, synthetic construct
  source 1..22: mol_type = other DNA
ctagaccggaaaagagaagcca

SEQ ID NO: 9 - DNA, 21 bp, synthetic construct
  source 1..21: mol_type = other DNA
atgctagaccggaaaagagaa

SEQ ID NO: 10 - DNA, 20 bp, synthetic construct
  source 1..20: mol_type = other DNA
caatgctagaccggaaaaga

SEQ ID NO: 11 - DNA, 21 bp, synthetic construct
  source 1..21: mol_type = other DNA
aagtcccaatgctagaccgga

SEQ ID NO: 12 - DNA, 20 bp, synthetic construct
  source 1..20: mol_type = other DNA
tctccgaagtcccaatgcta

SEQ ID NO: 13 - DNA, 18 bp, synthetic construct
  source 1..18: mol_type = other DNA
gagctctccgaagtccca

SEQ ID NO: 14 - DNA, 20 bp, synthetic construct
  source 1..20: mol_type = other DNA
agaacagtggagctctccga

SEQ ID NO: 15 - DNA, 20 bp, synthetic construct
  source 1..20: mol_type = other DNA
cgcccagaacagtggagctc

SEQ ID NO: 16 - DNA, 20 bp, synthetic construct
  source 1..20: mol_type = other DNA
cctcgcccagaacagtggag

SEQ ID NO: 17 - DNA, 21 bp, synthetic construct
  source 1..21: mol_type = other DNA
cagtggagctctccgaagtcc

SEQ ID NO: 18 - DNA, 21 bp, synthetic construct
  source 1..21: mol_type = other DNA
cccagaacagtggagctctcc

SEQ ID NO: 19 - DNA, 20 bp, synthetic construct
  source 1..20: mol_type = other DNA
cacagccctcgcccagaaca

SEQ ID NO: 20 - DNA, 20 bp, synthetic construct
  source 1..20: mol_type = other DNA
ttcttcacagccctcgccca

SEQ ID NO: 21 - DNA, 86 bp, synthetic construct
  source 1..86: mol_type = other DNA
tctttcttcacagccctcgcccagaacagtggagctctccgaagtcccaatgctagaccggaaaagagaagccaaaggagacctga

SEQ ID NO: 22 - DNA, 51 bp, synthetic construct
  source 1..51: mol_type = other DNA
tctccgaagtcccaatgctagaccggaaaagagaagccaaaggagacctga

SEQ ID NO: 23 - DNA, 52 bp, synthetic construct
  source 1..52: mol_type = other DNA
tctttcttcacagccctcgcccagaacagtggagctctccgaagtcccaatg

SEQ ID NO: 24 - DNA, 46 bp, synthetic construct
  source 1..46: mol_type = other DNA
ttcttcacagccctcgcccagaacagtggagctctccgaagtccca

SEQ ID NO: 25 - DNA, 20 bp, synthetic construct
  source 1..20: mol_type = other DNA
tgatagggaagcagggactg

SEQ ID NO: 26 - DNA, 20 bp, synthetic construct
  source 1..20: mol_type = other DNA
ggtgaggattgaggctgaaa

SEQ ID NO: 27 - DNA, 20 bp, synthetic construct
  source 1..20: mol_type = other DNA
cctttccaaagctgaactgg

SEQ ID NO: 28 - DNA, 20 bp, synthetic construct
  source 1..20: mol_type = other DNA
gccaatgcctgagagaagtc

SEQ ID NO: 29 - DNA, 20 bp, synthetic construct
  source 1..20: mol_type = other DNA
gtcagcactcacgttgccta

SEQ ID NO: 30 - DNA, 20 bp, synthetic construct
  source 1..20: mol_type = other DNA
cctataggactatccaggaa

SEQ ID NO: 31 - DNA, 20 bp, synthetic construct
  source 1..20: mol_type = other DNA
ccgagaatttgcttcagagg

SEQ ID NO: 32 - DNA, 20 bp, synthetic construct
  source 1..20: mol_type = other DNA
gcattaggtccaacccttga

SEQ ID NO: 33 - DNA, 20 bp, synthetic construct
  source 1..20: mol_type = other DNA
accgaccaggtaatcacagc

SEQ ID NO: 34 - DNA, 20 bp, synthetic construct
  source 1..20: mol_type = other DNA
ccataggaaggtgtgggaga

SEQ ID NO: 35 - DNA, 20 bp, synthetic construct
  source 1..20: mol_type = other DNA
ggggacaagatgtggagaga

(19) United States
(12) Patent Application Publication
(10) Pub. No.: US 2026/0327625 A1
(43) Pub. Date: Oct. 22, 2026

(54) ANTISENSE OLIGONUCLEOTIDES AS FRAGILE X SYNDROME THERAPEUTICS

(71) Applicant: UNIVERSITY OF MASSACHUSETTS, Westborough, MA (US)

(72) Inventors: Minggang FANG, Northborough, MA (US); Jonathan Kenneth WATTS, Worcester, MA (US); Michael Richard GREEN, Boylston, MA (US)

(21) Appl. No.: 19/491,553

(22) PCT Filed: Jun. 13, 2024
(86) PCT No.: PCT/US2024/033749

§ 371 Date: Dec. 9, 2025

Related U.S. Application Data
(60) Provisional application No. 63/508,134, filed on Jun. 14, 2023.

(51) Int. Cl. C12N 15/113; A61P 25/00

(52) U.S. Cl. CPC C12N 15/1137; A61P 25/00; C12Y 201/01043; C12N 2310/11; C12N 2310/311; C12N 2310/312; C12N 2310/313; C12N 2310/314; C12N 2310/315; C12N 2310/321; C12N 2310/322; C12N 2310/3233; C12N 2310/3341; C12N 2320/31

(57) ABSTRACT

Described herein is a EZH2 antisense oligonucleotide (ASO) epigenetic modulator of fragile X messenger ribonucleo-protein 1 (FMR1), its pharmaceutical compositions, and methods of treating a fragile X-associated disorder with the enhancer of zeste homolog 2 (EZH2) ASO. Also includes are pharmaceutical compositions including the EZH2 ASO and an ASO that decreases splicing between Exons 1 and 2 of FMRI-217. Methods of treating a fragile X-associated disorder with an EZH2 ASO and an ASO that decreases splicing between Exons 1 and 2 of FMR1-2F7 are also described.

### CROSS-REFERENCE TO RELATED APPLICATIONS

This Application claims priority to, and the benefit of, U.S. Provisional Patent Application No. 63/508,134, filed 14 Jun. 2023 and titled ANTISENSE OLIGONUCLEOTIDES AS FRAGILE X SYNDROME THERAPEUTICS, which is incorporated by reference herein in its entirety.

### STATEMENT REGARDING FEDERALLY SPONSORED RESEARCH & DEVELOPMENT

This invention was made with government support under NS111990, and MH113874 awarded by the National Institutes of Health. The government has certain rights in the invention.

### INCORPORATION BY REFERENCE

In compliance with 37 C.F.R. 1.52 (e), the sequence information contained in electronic file name UM30008PCT Sequence Listing ST26.xml, with a size of 33.4 KB and created on 20 May 2024, is incorporated by reference herein in its entirety.

FIELD OF THE DISCLOSURE

The present disclosure is related to antisense oligonucleotides targeting enhancer of zeste homolog 2 (EZH2) and optionally antisense oligonucleotides complementary to a portion of the pseudo exon of the fragile X messenger ribonucleoprotein 1 (FMR1) gene for the treatment of fragile X-associated disorders.

### BACKGROUND

Fragile X Syndrome (FXS) is the most common inherited form of intellectual disability and most prevalent monogenic cause of autism, occurring in approximately 1 in 4,000 males and approximately 1 in 8,000 females. Currently there are no curative therapies for FXS. The disease is caused by a CGG repeat expansion in the 5′ untranslated region of the X-linked fragile X messenger ribonucleoprotein 1 (FMR1) gene. Normal individuals have 6-54 repeats, whereas expansion of the repeats to >200 results in an FMR1 full mutation, which leads to transcriptional inactivation of FMR1 by a process referred to as epigenetic silencing. The epigenetically silenced FMR1 gene has the typical hallmarks of heterochromatin including DNA hypermethylation, gain of repressive histone modifications such as H3 lysine 9 trimethylation (H3K9me3), H3 lysine 27 trimethylation (H3K27me3) and H4 lysine 20 trimethylation (H4K20me3), and loss of activating histone modifications such as H3 lysine 4 trimethylation (H3K4me3) and H2A/H2B/H3/H4 acetylation. Although the epigenetic marks on the silenced FMR1 promoter are known, the specific factors that write, read or erase these marks are surprisingly poorly understood.

As a consequence of FMR1 silencing, the product of FMR1, the fragile X mental retardation protein (FMRP), is not produced. FMRP is a highly conserved protein expressed in all cells but is particularly prevalent in the brain. FMRP is an ribonucleic acid (RNA)-binding protein that predominantly functions by repressing mRNA translation, and in its absence protein synthesis is excessive, which results in disease pathology. In FXS neurons, the translation dysfunction results in several characteristic molecular abnormalities including increased levels of the neuronal-specific transcription repressor RE1-Silencing Transcription factor (REST), decreased expression of axonal guidance genes, and diminished levels of the signaling protein diacylglycerol kinase kappa (DGKK). FMRP has also been proposed to have other activities, including direct regulation of ion channels.

A prominent manifestation of FXS is synaptic weakening, which is measured electrophysiologically as long-term depression (LTD). A particular form of LTD, metabotropic glutamate receptor 5 (mGluR5)-LTD, is abnormally exaggerated in FXS, which has given rise to the so-called mGluR theory of FXS. This theory posits that inhibition of mGluR5 signaling should reverse or rescue pathophysiologies associated with the disease. In support of the mGluR theory, inhibition of mGluR5 signaling corrects disease symptomatology in Fmr1-/-knockout mice, the major animal model of FXS. However, large-scale clinical trials using mGluR5 antagonists have shown no beneficial effect in FXS patients. Thus, there is a great need for therapeutic approaches that are not based on mGluR5 inhibition.

Reactivation of the epigenetically silenced FMR1 gene is a potential therapeutic approach for FXS that would correct the root cause of the disease, the aberrant gene expression, rather than a secondary, downstream consequence of FMRP deficiency, such as increased mGluR5 signaling. Several considerations suggest that reactivation of epigenetically silenced FMR1 in FXS patients will result in increased expression of FMR1 and decreased disease pathology. First, targeted deletion or demethylation of the FMR1 CGG repeats in cultured FXS cells results in transcription reactivation of FMR1. Second, proof-of-principle studies in mouse and Drosophila models of FXS have shown that disease symptoms are reversible. Finally, there are rare asymptomatic individuals who have an FMR1 full mutation but still express FMR1, suggesting that restoration of FMR1 transcription in an FXS mutant background will ameliorate disease.

As described in WO 2017/049192 A1, to further explore the feasibility of the FMR1 reactivation approach, a candidate-based RNA interference (RNAi) screen was performed to identify epigenetic regulators that promote silencing of FMR1 in FXS cells. Using that approach, the histone methyltransferase enhancer of zeste homolog 2 (EZH2) was identified. Inhibition of EZH2 using an small hairpin RNA (shRNA) or small molecule inhibitor (EPZ6438/tazemetostat or GSK126) reactivates FMR1 in cultured undifferentiated induced pluripotent stem cells, neural progenitor cells (NPCs) and post-mitotic neurons derived from FXS patients. However, current EZH2 inhibitors do not efficiently cross the blood-brain barrier, which limits their potential use as FXS therapeutics.

Described herein are novel antisense oligonucleotides for the treatment of fragile X-associated disorders.

### BRIEF SUMMARY

In an aspect, an enhancer of zeste homolog 2 (EZH2) antisense oligonucleotide (ASO) epigenetic modulator of fragile X messenger ribonucleoprotein 1 (FMR1) has a length of 16-25 nucleotides and comprises a sequence at least 90% identical to GTCTACATGTTTTGGTCC (SEQ ID NO: 1) or TGTCTACATGTTTTGGTCC (SEQ ID NO: 2), wherein one or more T nucleotides is optionally replaced with U, and one or more C nucleotides is optionally replaced with 5-methyl C.

Also described is a pharmaceutical composition comprising the EZH2 antisense oligonucleotide epigenetic modulator of FMR1 and a pharmaceutically acceptable carrier. Methods of treating a fragile X-associated disorder in a patient in need of such treatment, comprising administering to a subject in need thereof the pharmaceutical composition are also included.

In another aspect, a pharmaceutical composition comprises the EZH2 antisense oligonucleotide epigenetic modulator of FMR1, an antisense oligonucleotide that decreases splicing between Exons 1 and 2 of FMR1-217, and a pharmaceutically acceptable carrier.

In yet another aspect, a method of treating a fragile X-associated disorder in a patient in need of such treatment comprises administering to the subject an EZH2 antisense oligonucleotide epigenetic modulator of FMR1 having a length of 16-25 nucleotides, and simultaneously or sequentially administering to the subject an antisense oligonucleotide that decreases splicing between Exons 1 and 2 of FMR1-217 which is complementary to a sequence that is at least 90% identical to 18-29 contiguous nucleotides of SEQ ID NO: 5.

### BRIEF DESCRIPTION OF THE DRAWINGS

FIGS. 1A, 1B, 1C, and 1D show derivation of enhancer of zeste homolog 2 (EZH2) antisense oligonucleotides (ASOs) that reactivate epigenetically silenced fragile X messenger ribonucleoprotein 1 (FMR1) in FXS neurons. (1A) quantitative reverse transcription polymerase chain reaction (qRT-PCR) analysis monitoring EZH2 expression in FXS 848-neurons treated with a control or EZH2 ASO. (1B) Immunoblot monitoring levels of H3 lysine 27 trimethylation (H3K27me3) in FXS 848-neurons expressing an EZH2 ASO or a control ASO. The results confirm that EZH2 ASOs reduce the levels of H3K27me3 in human cells. (1C) qRT-PCR analysis monitoring FMR1 expression in cultured FXS 848-neurons treated with a control or EZH2 ASO. (1D) qRT-PCR analysis monitoring FMR1 in FXS (CGG-intact)-neurons treated with a control ofrEZH2 ASO. The results were normalized to that obtained in FXS (CGG-excised)-neurons, which was set to 1. Data are represented as mean±SD (n=3 biological replicates). * P<0.05, ** P<0.01.

FIGS. 2A, 2B, 2C, 2D, 2E, and 2F show EZH2 ASOs normalize characteristic molecular abnormalities of FXS neurons. (2A,2B) qRT-PCR analysis monitoring expression of RE1-Silencing Transcription factor (REST) (2A) or deleted in colorectal carcinoma or netrin receptor DCC (DCC), roundabout homolog 3 (ROB03) and slit homolog 1 protein (SLIT1) (2B) in cultured FXS 848-neurons treated with an EZH2 ASO. (2C,2D) qRT-PCR analysis monitoring REST (2C) or DCC, ROB03 and SLIT1 (2D) in FXS (CGG-intact)-neurons treated with a control of EZH2 ASO. The results were normalized to that obtained in FXS (CGG-excised)-neurons, which was set to 1. The diacylglycerol kinase kappa (DGKK) signal was quantified relative to that obtained in normal neurons. (2E) Immunoblot analysis showing DGKK levels in cultured FXS 848-neurons treated with a control or EZH2 ASO. (2F) Immunoblot analysis showing DGKK levels in cultured FXS (CGG-intact)-neurons treated with a control or EZH2 ASO. Data are represented as mean ±SD (n=3 biological replicates). *P<0.05, **P<0.01.

FIGS. 3A and 3B show EZH2 ASOs correct electrophysiological abnormalities in cultured FXS neurons. (3A) Multielectrode Array (MEA) showing firing frequency of cultured FXS 848-neurons treated with an EZH2 ASO. The firing frequency of normal neurons is shown. (3B) MEA showing firing frequency of cultured FXS (CGG-intact)-neurons treated with a control or EZH2 ASO. The firing frequency of FXS (CGG-excised) neurons is shown. Data are represented as mean ±SD (n=3 biological replicates). *P<0.05, ** P<0.01.

FIGS. 4A and 4B show EZH2 ASOs reactivate FMR1 expression in human FXS neural progenitor cells (NPCs) engrafted within the brains of mice. (4A) qRT-PCR analysis monitoring FMR1 expression in FXS 848-NPC grafts in mice (n=4) treated with an EZH2 or control ASO by intracerebroventricular (ICV) injection. (4B) Representative immunohistochemical images of mouse brain sections (subventricular zone) showing staining for human FMRP (green), human mitochondria (red) and 4′,6-diamidino-2-phenylindole (DAPI; blue) following treatment with an EZH2 or control ASO. The merged image is shown. Data are represented as mean ±SD (n=4 biological replicates). **P<0.01.

The above-described and other features will be appreciated and understood by those skilled in the art from the following detailed description, drawings, and appended claims.

### DETAILED DESCRIPTION

Described herein are enhancer of zeste homolog 2 (EZH2) antisense oligonucleotides (ASOs) that reactivate fragile X messenger ribonucleoprotein 1 (FMR1) expression and substantially correct characteristic molecular and electrophysiological abnormalities in cultured fragile X syndrome (FXS) neurons, and reactivate FMR1 in human FXS neural progenitor cells (NPCs) engrafted within the brains of mice. ASO therapeutics have received considerable attention for treatment of certain central nervous system (CNS) disorders, based largely on the success of the intrathecal administration of the ASO nusinersen to treat spinal muscular atrophy. ASOs have a number of attractive features as CNS therapeutics, including rapid distribution throughout the spinal cord and into most regions of the brain following intrathecal injection, and relatively long half-life in the CNS tissues allowing for infrequent administration. Collectively, the results establish EZH2 ASOs in particular, as a potential and feasible therapeutic approach for FXS.

Previous studies have shown that deoxyribonucleic acid (DNA) demethylation can reactivate epigenetically silenced FMR1. For example, targeted demethylation of the FMR1 CGG repeats by a dCas9-Tet1 fusion protein was found to reactivate FMR1 . However, from a clinical perspective it is currently not feasible, by gene therapy or other approaches, to deliver the large dCas9-Tet1 fusion protein to a sufficient number of CNS neurons to have a therapeutic benefit. In addition, several previous studies have shown reactivation of FMR1 by DNA methyltransferase (DNMT) inhibitors such as 5-aza-2′-deoxycytidine (5-aza-dC) in cultured cells and following systemic treatment of mice in the human NPC mouse brain engraftment model. However, 5-aza-dC is poorly CNS penetrant and has significant toxicity. For example, following systemic administration, the concentration of 5-aza-dC in the peripheral circulation is approximately 100-fold higher than that in the CNS, which would result in unacceptable toxicity. EZH2 ASOs represent a modality that can be used to reactivate FMR1 as an alternative to toxic FMR1 reactivation therapies such as 5-aza-dC.

As used herein an ASO is a polynucleotide comprising a nucleotide sequence that is complementary (e.g., fully complementary or partially complementary) to a target sequence described herein (such that the polynucleotide is capable of hybridizing or annealing to target sequence, e.g., under physiological conditions). As used herein, “complementary” refers to sequence complementarity between two different polynucleotides or between two regions of the same polynucleotide. A first region of a polynucleotide is complementary to a second region of the same or a different polynucleotide if, when the two regions are arranged in an anti-parallel fashion, at least one nucleotide residue of the first region is capable of base pairing (i.e., hydrogen bonding) with a residue of the second region, thus forming a hydrogen-bonded duplex. An ASO can be an antisense oligodeoxynucleotide or an antisense oligoribonucleotide, or mixed oligodeoxynucleotide and oligoribonucleotide.

In any of the ASOs disclosed herein, each nucleobase shown as T may independently be T or U. Similarly, each C nucleotide may independently be C or a C analogue such as 5-methyl C, or other substituted C analogue. Other modified nucleobases with equivalent Watson-Crick base pairing properties will be known to one of skill in the art and would also be appropriate for use in the ASOs.

As used herein, the term “sequence identity,” refers to the extent to which two nucleotide sequences have the same residues at the same positions when the sequences are aligned to achieve a maximal level of identity, expressed as a percentage. For sequence alignment and comparison, typically one sequence is designated as a reference sequence, to which a test sequences are compared. Sequence identity between reference and test sequences is expressed as a percentage of positions across the entire length of the reference sequence where the reference and test sequences share the same nucleotide or amino acid upon alignment of the reference and test sequences to achieve a maximal level of identity. As an example, two sequences are considered to have 90% sequence identity when, upon alignment to achieve a maximal level of identity, the test sequence has the same nucleotide residue at 90% of the same positions over the entire length of the reference sequence.

Alignment of sequences for comparison to achieve maximal levels of identity can be readily performed by a person of ordinary skill in the art using an appropriate alignment method or algorithm. In some instances, alignment can include introduced gaps to provide for the maximal level of identity. Examples of sequence alignment tools are well- known in the art.

### EZH2 ASOs

In an aspect, described herein are EZH2 ASO epigenetic modulators of FMR1. Enhancer of Zeste Homolog 2 (EZH2) is the catalytic core component of the Polycomb Repressive Complex 2 (PRC2), which is responsible for gene silencing through trimethylation of H3K27. EZH2 promotes silencing of FMR1 in FXS cells, and it has been shown that inhibition of EZH2 using an shRNA or small molecule inhibitor reactivates FMR1 in cultured undifferentiated induced pluripotent stem cells, neural progenitor cells (NPCs) and post-mitotic neurons derived from FXS patients (WO 2017/049192 A1). EZH2 has NCBI RefSeq accession number NG_032043.1.

In an aspect, an EZH2 ASO epigenetic modulator of FMR1 has a length of 16-25 nucleotides and is complementary to a sequence which is at least 90% (e.g., at least 91%, at least 92%, at least 93%, at least 94%, at least 95%, at least 96%, at least 98%, or 100%) identical to 16-25 contiguous nucleotides of the EZH2 gene.

In some aspects, the ASO is complementary to an intron of EZH2 . In some embodiments, the ASO is complementary to an exon of EZH2 messenger ribonucleic acid (mRNA).

In an aspect, an EZH2 ASO epigenetic modulator of FMR1 has a length of 16-25 nucleotides and comprises a sequence at least 90% (e.g., at least 91%, at least 92%, at least 93%, at least 94%, at least 95%, at least 96%, at least 98%, or 100%) identical to GTCTACATGTTTTGGTCC (SEQ ID NO: 1) or TGTCTACATGTTTTGGTCC (SEQ ID NO: 2). One of skill in the art will recognize that U may replace T in one or more positions; likewise 5-methyl C may replace C and similar nucleobase analogues may be incorporated.

In an aspect, the EZH2 ASO epigenetic modulator of FMR1 comprises a sequence at least 92%, at least 95%, at least 98%, or 100% identical to SEQ ID NO: 1 or 2.

In another aspect, the EZH2 ASO epigenetic modulator of FMR1 has a length of 18 to 22 nucleotides, specifically 18-20 nucleotides.

Chemical modifications of the ASOs can be chosen to, e.g., increase nuclease resistance of a polynucleotide (e.g., oligonucleotide), to prevent RNase H cleavage of a polynucleotide (e.g., a complementary messenger ribonucleic acid (RNA) strand), or to increase cellular uptake of a polynucleotide. For each of these goals, a variety of compatible chemical modifications are available and will be familiar to those skilled in the art.

In an aspect, the EZH2 ASO epigenetic modulator of FMR1 comprises a modification of a ribose group such as 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH2, a constrained nucleotide, a tricyclo-DNA modification, a morpholino nucleotide, or a combination thereof. In an aspect, the ASO comprises one or more 2′-O-(2-methoxyethyl) (MOE) modifications.

In another aspect, the ASO epigenetic modulator of FMR1 comprises a modification of a phosphate group and wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof. In an aspect, the ASO comprises one or more phosphorodithioate modifications.

In a specific aspect, the EZH2 ASO epigenetic modulator of FMR1 of SEQ ID NO: 1 comprises &G*&T&C&T&A*C*A*T*G*T*T*T*T*&G&G&T&C*&C, or SEQ ID NO: 2 comprises &T*&G&T&C&T*A*C*A*T*G*T*T*T*T*&G&G&T&C*&C, wherein & is a 2′-deoxy, 2′-O-(2-methoxyethyl) modification and * is a phosphorothioate linkage.

In an aspect, a pharmaceutical composition comprises the EZH2 ASO epigenetic modulator of FMR1 and a pharmaceutically acceptable carrier.

In another aspect, a method of treating a fragile X-associated disorder in a patient in need of such treatment comprises administering to a subject in need thereof the pharmaceutical composition comprising the EZH2 ASO epigenetic modulator of FMR1. Exemplary fragile X-associated disorders include fragile X syndrome (FXS), fragile X-associated primary ovarian insufficiency (FXPOI), or fragile X-associated tremor/ataxia syndrome (FXTAS).

### ASOs Complimentary to a Portion of the Pseudo Exon of the FMR1 Gene

It has been found that 50 to 70% of FXS patients have background expression of FMR1 mRNA, which is mostly a mis-spliced isoform that does not lead to productive fragile X messenger ribonucleoprotein (FMRP) protein. The mis-splicing can be corrected using ASOs leading to increased levels of the FMR1 gene product, the FMRP protein. Specifically, ASO treatment reduces the expression of the CGG expansion-dependent aberrantly spliced FMR1-217 RNA and restores FMRP to levels observed in cells from typically developing individuals.

In an aspect, the EZH2 ASO epigenetic modulator of FMR1 can be combined with an ASO that decreases splicing between Exons 1 and 2 of FMR1-217 and thus decreases expression of the protein encoded by FMR1-217.

In humans, FMR1-217, also referred to as “isoform 12” or “iso12,” is transcript corresponding to A0A087X1M7 (ENST00000621447.1, 1,832 nucleotides). FMR1-217 has 2 exons, and the splicing between Exon 1 of FMR1-217 (between base pairs 147,912,123 and 147,912,230, SEQ ID NO: 3) and Exon 2 of FMR1-217 (between base pairs 147,912,728 and 147,914,451, SEQ ID NO: 4) is considered aberrant FMR1 RNA splicing. FMR1-217 is detected in a subpopulation of subjects with fragile X-associated disorder, including a subpopulation of FXS patients, and a subpopulation of premutation carriers for FXS.

(SEQ ID NO: 3)
CGCCCGCAGCCCACCTCTCGGGGGCGGGCTCCCGGCGCTAGCAGGGCTGA
AGAGAAGATGGAGGAGCTGGTGGTGGAAGTGCGGGGCTCCAATGGCGCTT
TCTACAAG.
(SEQ ID NO: 4)
CATTGGGACTTCGGAGAGCTCCACTGTTCTGGGCGAGGGCTGTGAAGAAA
GAGTAGTAAGAAGCGGTAGTCGGCACCAAATCACAATGGCAACTGATTTT
TAGTGGCTTCTCTTTGTGGATTTCGGAGGAGATTTTAGATCCAAAAGTTT
CAGGAAGACCCTAACATGGCCCAGCAGTGCATTGAAGAAGTTGATCATCG
TGAATATTCGCGTCCCCCTTTTTGTTAAACGGGGTAAATTCAGGAATGCA
CATGCTTCAGCGTCTAAAACCATTAGCAGCGCTGCTACTTAAAAATTGTG
TGTGTGTGTTTAAGTTTCCAAAGACCTAAATATATGCCATGAAACTTCAG
GTAATTAACTGAGAGTATATTATTACTAGGGCATTTTTTTTTTAACTGAG
CGAAAATATTTTTGTGCCCCTAAGAACTTGACCACATTTCCTTTGAATTT
GTGGTGTTGCAGTGGACTGAATTGTTGAGGCTTTATATAGGCATTCATGG
GTTTACTGTGCTTTTTAAAGTTACACCATTGCAGATCAACTAACACCTTT
CAGTTTTAAAAGGAAGATTTACAAATTTGATGTAGCAGTAGTGCGTTTGT
TGGTATGTAGGTGCTGTATAAATTCATCTATAAATTCTCATTTCCTTTTG
AATGTCTATAACCTCTTTCAATAATATCCCACCTTACTACAGTATTTTGG
CAATAGAAGGTGCGTGTGGAAGGAAGGCTGGAAAATAGCTATTAGCAGTG
TCCAACACAATTCTTAAATGTATTGTAGAATGGCTTGAATGTTTCAGACA
GGACACGTTTGGCTATAGGAAAATAAACAATTGACTTTATTCTGTGTTTA
CCAATTTTATGAAGACATTTGGAGATCAGTATATTTCATAAATGAGTAAA
GTATGTAAACTGTTCCATACTTTGAGCACAAAGATAAAGCCTTTTGCTGT
AAAAGGAGGCAAAAGGTAACCCCGCGTTTATGTTCTTAACAGTCTCATGA
ATATGAAATTGTTTCAGTTGACTCTGCAGTCAAAATTTTAATTTCATTGA
TTTTATTGATCCATAATTTCTTCTGGTGAGTTTGCGTAGAATCGTTCACG
GTCCTAGATTAGTGGTTTTGGTCACTAGATTTCTGGCACTAATAACTATA
ATACATATACATATATATGTGTGAGTAACGGCTAATGGTTAGGCAAGATT
TTGATTGACCTGTGATATAAACTTAGATTGGATGCCACTAAAGTTTGCTT
ATCACAGAGGGCAAGTAGCACATTATGGCCTTGAAGTACTTATTGTTCTC
TTCCAGCAACTTATGATTTGCTCCAGTGATTTTGCTTGCACACTGACTGG
AATATAAGAAATGCCTTCTATTTTTGCTATTAATTCCCTCCTTTTTTGTT
TTGTTTTGTAACGAAGTTGTTTAACTTGAAGGTGAATGAAGAATAGGTTG
GTTGCCCCTTAGTTCCCTGAGGAGAAATGTTAATACTTGAACAAGTGTGT
GTCAGACAAATTGCTGTTATGTTTATTTAATTAAGTTTGATTTCTAAGAA
AATCTCAAATGGTCTGCACTGATGGAAGAACAGTTTCTGTAACAAAAAAG
CTTGAAATTTTTATATGACTTATAATACTGCTGTGAGTTTTAAAAGTAAA
GCAAAAGTAAACTGAGTTGCTTGTCCAGTGGGATGGACAGGAAAGATGTG
AAATAAAAACCAATGAAAAATGAA

In an aspect, an ASO that decreases splicing between Exons 1 and 2 of FMR1-217 is complementary to a sequence that is at least 90% (e.g., at least 91%, at least 92%, at least 93%, at least 94%, at least 95%, at least 96%, at least 98%, or 100%) identical to 18-29 contiguous nucleotides of SEQ ID NO: 5.

(SEQ ID NO: 5)
AGCGGGGGGCGCTTCAGCCGGGCCGCCTCCTGCAGCGCCAAGAGGGCTTC
AGGTCTCCTTTGGCTTCTCTTTTCCGGTCTAGCATTGGGACTTCGGAGAG
CTCCACTGTTCTGGGCGAGGGCTGTGAAGAAAGAGTAGTAAGAAGCGGTA
GTCGGCACCAAATCACAATGGCAACTGATTTTTAGTGGCTTCTCTTTGTG
GATTTCGGAGGAGATTTTAGATCCAAAAGTTTCAGGAAGACCCTAACATG
GCCCAGCAGTGCATTGAAGAAGTTGATCATCGTGAATATTCGCGTCCCCC
TTTTTGTTAAACGGGGTAAATTCAGGAATGCACATGCTTCAGCGTCTAAA
ACCATTAGCAGCGCTGCTACTTAAAAATTGTGTGTGTGTGTTTAAGTTTC
CAAAGACCTAAATATATGCC

In an aspect, the ASO that decreases splicing between Exons 1 and 2 of FMR1-217 is complementary to a sequence that is at least 92%, at least 95%, at least 98% or 100% identical to 18-29 contiguous nucleotides of SEQ ID NO: 5.

In another aspect, the ASO that decreases splicing between Exons 1 and 2 of FMR1-217 has a length of 18-29 nucleotides and comprises is at least 90%, at least 92%, at least 95%, at least 98% or 100% identical to any of SEQ ID NOs. 6-24.

(W-704)
(SEQ ID NO: 6)
AGAAGCCAAAGGAGACCTGA.
(W-705)
(SEQ ID NO: 7)
AAAGAGAAGCCAAAGGAGAC.
(W-706)
(SEQ ID NO: 8)
CTAGACCGGAAAAGAGAAGCCA.
(W-707)
(SEQ ID NO: 9)
ATGCTAGACCGGAAAAGAGAA.
(W-708)
(SEQ ID NO: 10)
CAATGCTAGACCGGAAAAGA.
(W-709)
(SEQ ID NO: 11)
AAGTCCCAATGCTAGACCGGA.
(W-710)
(SEQ ID NO: 12)
TCTCCGAAGTCCCAATGCTA.
(W-711)
(SEQ ID NO: 13)
GAGCTCTCCGAAGTCCCA.
(W-712)
(SEQ ID NO: 14)
AGAACAGTGGAGCTCTCCGA.
(W-713)
(SEQ ID NO: 15)
CGCCCAGAACAGTGGAGCTC.
(W-714)
(SEQ ID NO: 16)
CCTCGCCCAGAACAGTGGAG.
(2831)
(SEQ ID NO: 17)
CAGTGGAGCTCTCCGAAGTCC.
(2832)
(SEQ ID NO: 18)
CCCAGAACAGTGGAGCTCTCC.
(2833)
(SEQ ID NO: 19)
CACAGCCCTCGCCCAGAACA.
(2834)
(SEQ ID NO: 20)
TTCTTCACAGCCCTCGCCCA.
(SEQ ID NO: 21)
TCTTTCTTCACAGCCCTCGCCCAGAACAGTGGAGCTCTCCGAAGTC
CCAATGCTAGACCGGAAAAGAGAAGCCAAAGGAGACCTGA.
(SEQ ID NO: 22)
TCTCCGAAGTCCCAATGCTAGACCGGAAAAGAGAAGCCAAAGGAG
ACCTGA.
(SEQ ID NO: 23)
TCTTTCTTCACAGCCCTCGCCCAGAACAGTGGAGCTCTCCGAAGTC
CCAATG.
(SEQ ID NO: 24)
TTCTTCACAGCCCTCGCCCAGAACAGTGGAGCTCTCCGAAGTCCCA.

In any of SEQ ID NOs. 6 to 24, one or more T nucleotides is optionally replaced with U, and one or more C nucleotides is optionally replaced with 5-methyl C.

In an aspect, the ASO that decreases splicing between Exons 1 and 2 of FMR1-217 comprises a modification of a ribose group such as 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH2, a constrained nucleotide, a tricyclo-DNA modification, a morpholino nucleotide, or a combination thereof. In an aspect, the ASO comprises one or more 2′-O-(2-methoxyethyl) (MOE) modifications.

In another aspect, the ASO that decreases splicing between Exons 1 and 2 of FMR1-217 comprises a modification of a phosphate group and wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof. In an aspect, the ASO comprises one or more phosphorodithioate modifications.

### Pharmaceutical Compositions

In an aspect, a pharmaceutical composition comprises the EZH2 antisense oligonucleotide epigenetic modulator of FMR1 and a pharmaceutically acceptable carrier.

In another aspect, a pharmaceutical composition comprises the EZH2 antisense oligonucleotide epigenetic modulator of FMR1, the antisense oligonucleotide that decreases splicing between Exons 1 and 2 of FMR1-217, and a pharmaceutically acceptable carrier.

In an aspect, an ASO is in a form of a pharmaceutical composition, or a pharmaceutically acceptable salt thereof. A “pharmaceutical composition” refers to a formulation of one or more therapeutic agents and a medium generally accepted in the art for delivery of a biologically active agent to subjects, e.g., humans. In some embodiments, a pharmaceutical composition may include one or more pharmaceutically acceptable excipients, diluents, or carriers. “Pharmaceutically acceptable carrier, diluent, or excipient” includes any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In some embodiments, a pharmaceutical composition disclosed herein is formulated as a solution.

“Pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative. In some embodiments, the carrier may be a diluent, adjuvant, excipient, or vehicle with which the ASO is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine can be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, and the like. The concentration of the agent in such pharmaceutical formulation may vary widely, i.e., from less than about 0.5%, to at least about 1%, or to as much as 15%, 20%, 25%, 30%, 35%, 40%, 45% or 50% by weight. The concentration will be selected primarily based on required dose, fluid volumes, viscosities, and the like, according to the mode of administration.

Non-limiting examples of pharmaceutically acceptable carriers are solvents, dispersion media, coatings, antibacterial and antifungal agents, isotonic and absorption delaying agents, and the like that are physiologically compatible, such as salts, buffers, antioxidants, saccharides, aqueous or non-aqueous carriers, preservatives, wetting agents, surfactants or emulsifying agents, or combinations thereof.

Non-limiting examples of buffers that may be used are acetic acid, citric acid, formic acid, succinic acid, phosphoric acid, carbonic acid, malic acid, aspartic acid, histidine, boric acid, Tris buffers, N-(2-Hydroxyethyl) piperazine-N′-(2-hydroxypropanesulphonic acid (HEPPSO) and 4-(2-hydroxyethyl)-1-piperazineethanesulfonic acid (HEPES).

Non-limiting examples of antioxidants that may be used are ascorbic acid, methionine, cysteine hydrochloride, sodium bisulfate, sodium metabisulfite, sodium sulfite, lecithin, citric acid, ethylenediamine tetraacetic acid (EDTA), sorbitol, and tartaric acid.

Non-limiting examples of amino acids that may be used are histidine, isoleucine, methionine, glycine, arginine, lysine, L-leucine, tri-leucine, alanine, glutamic acid, L-threonine, and 2-phenylamine.

Non-limiting examples of surfactants that may be used are polysorbates (e.g., polysorbate-20 or polysorbate-80); polyoxamers (e.g., poloxamer 188); Triton™; sodium octyl glycoside; lauryl-, myristyl-, linoleyl-, or stearyl-sulfobetaine; lauryl-, myristyl-, linoleyl-or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethyl glycol, polypropyl glycol, and copolymers of ethylene and propylene glycol (e.g., PLURONICST™, PF68, and the like.).

Non-limiting examples of preservatives that may be used are phenol, m-cresol, p-cresol, o-cresol, chlorocresol, benzyl alcohol, phenylmercuric nitrite, phenoxyethanol, formaldehyde, chlorobutanol, magnesium chloride, alkylparaben (methyl, ethyl, propyl, butyl and the like), benzalkonium chloride, benzethonium chloride, sodium dehydroacetate and thimerosal, or mixtures thereof.

Non-limiting examples of saccharides that may be used are monosaccharides, disaccharides, trisaccharides, polysaccharides, sugar alcohols, reducing sugars, nonreducing sugars such as glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol or iso-maltulose.

Non-limiting examples of salts that may be used are acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono-and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like. In some embodiments, the salt is sodium chloride (NaCl).

In some embodiments, an ASO is administered intravenously, intra-arterially, intrathecally, intraventricularly, intramuscularly, intradermally, subcutaneously, intracranially, or spinally. “Administering” or “administration” as used herein, refers to taking steps to deliver an agent to a subject, such as a mammal, in need thereof. Administering can be performed, for example, once, a plurality of times, and/or over one or more extended periods. Administration includes both direct administration, including self-administration, and indirect administration, including an act of prescribing a drug or directing a subject to consume an agent. For example, as used herein, one (e.g., a physician) who instructs a subject (e.g., a patient) to self-administer an agent (e.g., a drug), or to have an agent administered by another and/or who provides a patient with a prescription for a drug is administering an agent to a subject.

In some embodiments, an ASO is delivered locally to the central nervous system. This can include intrathecal or intraventricular injections, including the use of a catheter or Ommaya reservoir. Other methods of delivering drugs directly to the cerebrospinal fluid or central nervous system will be known to one skilled in the art.

In some embodiments, an ASO is administered as intrathecal bolus injection.

In some embodiments, an ASO is delivered systemically, such as via intravenous or subcutaneous injection. In some embodiments, the ASO is delivered using an approach that enhances bioavailability in the central nervous system after systemic administration. These approaches can include modification of the sugars or phosphate linkages, delivering as a duplex with a ligand-conjugated RNA molecule, formulation into an artificial exosome, liposome, polymer nanoparticle or lipid nanoparticle, or conjugation to lipids, antibodies, peptides, sugars, neuroactive molecules, or other moieties that enhance delivery to the central nervous system. In some embodiments, the agent (e.g., polynucleotide such as ASO) is delivered after transiently disrupting the blood-brain barrier. Other methods of enhancing bioavailability in the central nervous system after systemic administration will be known to one skilled in the art.

### Methods of Treatment

In an aspect, a method of treating a fragile X-associated disorder in a patient in need of such treatment comprises administering to a subject in need thereof a pharmaceutical composition comprising the EZH2 antisense oligonucleotide epigenetic modulator of FMR1 and a pharmaceutically acceptable carrier.

In another aspect, a method of treating a fragile X-associated disorder in a patient in need of such treatment comprises administering to a subject in need thereof a pharmaceutical composition comprising the EZH2 antisense oligonucleotide epigenetic modulator of FMR1, the antisense oligonucleotide that decreases splicing between Exons 1 and 2 of FMR1-217, and a pharmaceutically acceptable carrier.

In yet another aspect, a method of treating a fragile X-associated disorder in a patient in need of such treatment comprises administering to the subject an EZH2 antisense oligonucleotide epigenetic modulator of FMR1 having a length of 16-25 nucleotides, and simultaneously or sequentially administering to the subject an antisense oligonucleotide that decreases splicing between Exons 1 and 2 of FMR1-217 which is complementary to a sequence that is at least 90% (e.g., at least 91%, at least 92%, at least 93%, at least 94%, at least 95%, at least 96%, at least 98%, or 100%) identical to 18-29 contiguous nucleotides of SEQ ID NO: 5.

Fragile X-associated disorders are caused by mutation of the fragile X messenger ribonucleoprotein 1 (FMR1, previously known as fragile X mental retardation 1) gene, located in the q27.3 loci of the X chromosome. The expansion of the trinucleotide CGG above the normal range (greater than 54 repeats) in the non-coding region of the FMR1 gene has been associated with the development of fragile X-associated disorders. For example, in those carrying the premutation, the trinucleotide CGG can range from 55-200 CGG repeats. In some embodiments, a fragile X-associated disorder described herein is linked to greater than 77 CGG repeats in FMR1, e.g., greater than 98 CGG repeats in FMR1. In some embodiments, the fragile X-associated disorder is linked to at least 140 CGG repeats in FMR1. In some embodiments, the fragile X-associated disorder is linked to at least 200 CGG repeats in FMR1.

Non-limiting examples of fragile X-associated disorders include fragile-X associated tremor/ataxia syndrome (FXTAS), fragile X-associated primary ovarian insufficiency (FXPOI), fragile X-associated neuropsychiatric disorders (FXAND), and fragile X syndrome (FXS). In some embodiments, a fragile X-associated disorder described herein is fragile X syndrome (FXS), fragile X-associated primary ovarian insufficiency (FXPOI), or fragile X-associated tremor/ataxia syndrome (FXTAS), or a combination thereof. In some embodiments, the fragile X-associated disorder is FXS.

The term “subject” refers to a mammalian subject, preferably human, diagnosed with or suspected of having a fragile X-associated disorder (e.g., FXS).

In some embodiments, the subject has a CGG repeat expansion between about 55 and about 200 repeats in the 5′ untranslated region of an FMR1 gene. In some embodiments, the subject has a CGG repeat expansion exceeding 200 repeats in the 5′ untranslated region of an FMR1 gene. In some embodiments, the subject has a CGG repeat expansion that is partially methylated. In some embodiments, the subject has a CGG repeat expansion that is fully methylated. In some embodiments, the subject has an increased level of isoform 12 of FMR1, a decreased level of isoform 1 of FMR1, or a combination thereof.

In some embodiments, the subject has one X chromosome and one Y chromosome. In some embodiments, the subject has two X chromosomes. In some embodiments, the subject has two X chromosomes and one Y chromosome. In some embodiments, the subject has one X chromosome and two Y chromosomes.

In some embodiments, the subject is a human male. In some embodiments the subject is human female.

In some embodiments, a subject is a human. In some embodiments, the human subject has, or is predisposed to have a fragile X-associated disorder. In some embodiments the human subject has, or is predisposed to have FXS, FXPOI, FXTAS, or a combination thereof. In some embodiments, the human subject has, or is predisposed to have FXS. In some embodiments, the subject is a human (e.g., about 50 years of age or older) who has, or is predisposed to have, FXTAS.

In some embodiments, the subject has one or more of the physical and/or medical features associated with a fragile X-associated disorder (e.g., FXS). Non-limiting examples of physical features associated with FXS include a long face, prominent ears and chin, arched palate, large testicles at puberty, low muscle tone, flat feet, and hyperextensible joints. Non-limiting examples of medical or behavioral features associated with FXS include sleep problems, seizures, recurrent ear infections, mitral valve prolapse, behaviors of hyperactivity, short attention span, hand biting or hand flapping, poor eye contact and social skills, shyness, anxiety, autism, epilepsy, aggression, delayed speech and/or motor development, repetitive speech, sensitivity to sensory stimulation (including a hypersensitivity to being touched, to light or to sound). In some embodiments, the subject is a female with an intelligence quotient (IQ) score of less than 115, 110, 105, 100, 95 or 90. In some embodiments, the subject is a male with an IQ score of less than 60, 55, 50 or 45.

In some embodiments, the subject has one or more of the following: irregular menses, fertility problem, elevated follicle-stimulating hormone (FSH) level, premature ovarian failure, primary ovarian insufficiency, and vasomotor symptoms (e.g., “hot flash”). In some embodiments, the subject has one or more of the following: intention tremor, parkinsonism, ataxia, memory loss, white matter lesion involving middle cerebellar peduncles, and cognitive decline.

“Treat,” “treating” or “treatment” refers to therapeutic treatment wherein the objective is to slow down (lessen) an undesired physiological change or disease, such as the development or progression of the fragile X-associated disorder (e.g., FXS), or to provide a beneficial or desired clinical outcome during treatment. Beneficial or desired clinical outcomes include alleviation of symptoms, diminishment of extent of disease, stabilized (i.e., not worsening) state of disease, delay or slowing of disease progression, amelioration or palliation of the disease state, whether detectable or undetectable.

In some embodiments, treating a fragile X-associated disorder (e.g., FXS) includes slowing progression of the fragile X-associated disorder (e.g., FXS), alleviating one or more signs or symptoms of the fragile X-associated disorder (e.g., FXS), preventing one or more signs or symptoms of the fragile X-associated disorder (e.g., FXS), or a combination thereof.

Non-limiting examples of treatment benefits include improvements in speech and motor development; a reduction in or prevention of cognitive disabilities, ranging from learning disabilities to intellectual disability; alleviating or preventing physical and medical features such as a long face, prominent ears and chin, arched palate, large testicles at puberty, low muscle tone, flat feet, hyperextensible joints, sleep problems, seizures, recurrent ear infections, and mitral valve prolapse; reducing or preventing behaviors of hyperactivity, short attention span, hand biting or hand flapping, poor eye contact and social skills, shyness, anxiety, delayed speech and/or motor development, repetitive speech, and/or sensitivity to sensory stimulation (including a hypersensitivity to being touched).

In some embodiments, treatment may include modulation of or improvement in language, fragile X behaviors, brain activity, clinical impression, inattention, safety, social avoidance, cognition, hyperactivity, executive function, irritability, eye contact, or memory.

In still other embodiments, treatment can include reducing or preventing absent or irregular menses, fertility problems, elevated follicle-stimulating hormone (FSH) levels, premature ovarian failure, primary ovarian insufficiency, and/or hot flashes. In still further embodiments, treating may include reducing or preventing intention tremors, parkinsonism, ataxia, memory loss, white matter lesions involving middle cerebellar peduncles, and/or cognitive decline. In some embodiments, treatment may reduce or prevent neuropathy of extremities, mood instability, irritability, explosive outbursts, personality changes, autonomic function problems such as impotence, loss of bladder or bowel functions. Treatment may also include reducing or preventing high blood pressure, thyroid disorders, or fibromyalgia.

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual.

The invention is further illustrated by the following non-limiting examples.

### Examples

### Methods

Cell culture: FXS 848-iPS3 cells (FXS 848-iPSCs) were kindly provided by Stephen J. Haggarty (Harvard Medical School). The isogenic FXS (CGG-excised/intact) iPSC lines were kindly provided by Peng Jin (Emory University School of Medicine). Normal and FXS induced pluripotent stem cells (iPSCs) and NPCs were authenticated by quantitative reverse transcription polymerase chain reaction (qRT-PCR) analysis to validate the expected FMR1 expression status, and by polymerase chain reaction (PCR) to confirm the expected CGG repeat length in the FMR1 5′ untranslated regions (UTR). Upon receipt, cells were tested for mycoplasma contamination and found to be negative. iPSCs were cultured in mTeSR™1 medium (STEMCELL Technologies; Vancouver, Canada) on Matrigel® (Corning; Corning, New York)-coated plates. NPCs were maintained in neural expansion medium as described in the art. Neurons were induced from normal iPSCs or FXS 848-iPSCs by expression of reverse tetracycline transactivator (rTA3), Neurogenin-1 and Neurogenin-2, and cultured in mTeSR1 and neuron growth medium as described in the art.

For ASO treatment, FXS neurons were incubated with 1 μM control or EZH2 ASO for 3 weeks, and 80% of the medium (with ASO) was refreshed every 7 days.

qRT-PCR: Total RNA was isolated, and reverse transcription was performed as described in the art, followed by real-time qRT-PCR using Platinum® SYBR® Green qPCR SuperMix-UDG with Rox (Invitrogen; Waltham, Massachusetts). Gene-specific primers are listed in Table 1. The Taqman® assay was performed using Taqman® Fast Advanced Master Mix (ThermoFisher; Waltham, Massachusetts) and probes for FMR1 (Hs00924547_m1) and ACTB (Hs99999903_m1), all from Thermo Fisher, according to the manufacturer's instructions. The experiments were performed in biological triplicate, each with three technical triplicates.

TABLE 1
LIST OF QRT-PCR PRIMERS

| | Forward primer | Reverse primer |
|---|---|---|
| Gene | sequence (5′→3′) | sequence (5′→3′) |
| EZH2 | TGATAGGGAAGCAGGGACTG | CCGAGAATTTGCTTCAGAGG |
| | (SEQ ID NO: 25) | (SEQ ID NO: 31) |
| FMRI | GGTGAGGATTGAGGCTGAAA | GCATTAGGTCCAACCCTTGA |
| | (SEQ ID NO: 26) | (SEQ ID NO: 32) |
| REST | CCTTTCCAAAGCTGAACTGG | ACCGACCAGGTAATCACAGC |
| | (SEQ ID NO: 27) | (SEQ ID NO: 33) |
| ROBO3 | GCCAATGCCTGAGAGAAGTC | CCATAGGAAGGTGTGGGAGA |
| | (SEQ ID NO: 28) | (SEQ ID NO: 34) |
| SLIT1 | GTCAGCACTCACGTTGCCTA | GGGGACAAGATGTGGAGAGA |
| | (SEQ ID NO: 29) | (SEQ ID NO: 35) |

Immunoblotting: Protein extracts were prepared by lysis in a buffer containing 50 mM Tris-HCl (pH 7.4), 0.1% Triton™ X-100, 5 mM EDTA, 250 mM NaCl, 50 mM NaF, 0.1 mM Nₐ₃ VO₄, and protease inhibitors (Roche). Blots were probed with an antibody recognizing diacylglycerol kinase kappa (DGKK) (Abgent; San Diego, California), α-tubulin (Sigma), EZH2 (Bethyl) or H3K27me3 (Cell Signaling; Danvers, Massachusetts). The DGKK signals were quantified using Image J software (National Institutes of Health; Bethesda, Maryland), and normalized to α-tubulin levels as previously described in the art.

Multielectrode Array (MEA) assay: Neurons (at day 4 of the differentiation process) were seeded at 1x10⁵ cells/well in 6-well plates at 40% confluency, and incubated with 1 μM control or EZH2 ASO, and 80% of the medium (with ASO) was refreshed every 8 days. At day 8 of the differentiation process, MEA assays were performed every 4 days using a MED64 Presto MEA system (Alpha MED Scientific Inc.; Osaka, Japan) according to the manufacturer's instructions. Recordings of spontaneous activities were performed using a 3-kHz two-pole Butterworth low pass filter. Experiments were performed in biological triplicate.

Animal experiments. All mouse studies were performed in accordance with the Guide for the Care and Use of Laboratory Animals from NIH, and protocols (A2060) approved by the University of Massachusetts Chan Medical School (UMMS) Institutional Animal Care and Use Committee (IACUC). Mice were randomly allocated to each group. No blinding was done as animal groups were identified by tagging and labeling the cages with the cells/ASOs injected.

C57BL/6J mice (aged 6-9 months, n=4 per group) were anesthetized and placed in a rodent stereotaxic frame (Stoelting; Wood Dale, Illinois), and intracerebroventricular (ICV) injections were performed as previously described in the art. Briefly, the skull was exposed by a small longitudinal incision (<1 cm) along the midline, the periosteum was removed from the surgical area, and small burr holes (<1 mm) were made in each hemisphere using a high-speed drill at the stereotaxic coordinates for ICV injection (X: +/−mm; Y:-0.4 mm; Z:-1.6 mm). NPCs (5x10⁴ in 5 μl volume) were injected slowly into each site using an UltramicroPump (World Precision Instruments; Sarasota, Florida) to drive a Hamilton Syringe attached to a 31-gauge steel needle (Hamilton; Reno, Nevada). Staples were used to close the incision and mice were allowed to recover. Three days after NPC transplantation, the initial incision was reopened and 30 pM control or EZH2 ASO was injected into the same site. Seven days after the treatment, mice were sacrificed and the subventricular region was manually dissected for RNA and immunohistochemistry analysis. For RNA analysis, the tissue was mechanically disrupted, and RNA was extracted using a NucleoSpin® RNA Plus Kit (Macherey Nagel; Düren, Germany) and analyzed for FMR1 expression using primers specific for the human FMR1 gene.

For immunohistochemical staining, brain tissues were fixed in paraformaldehyde, embedded in paraffin and sectioned at a thickness of 10 μm. The immunolabeling was performed by the Morphology Core at the University of Massachusetts Chan Medical School. Briefly, following deparaffinization (60 °C for 30 min, xylene for 10 min, 100% ethanol 20 dips, 95% ethanol 10 dips, 75% ethanol 10 dips, H₂O 1 min), brain sections were processed for antigen retrieval with citrate acid (pH 6.0), blocked with 5% milk in TBS (wt/vol, pH 7.4) at 23-25 °C and incubated at 4 °C overnight in TBS containing 5% milk with or a mouse antibody to human FMRP (AbFrontier, 1:500) or a rabbit monoclonal antibody to human mitochondria (Millipore, 1:200). The next day, sections were washed in PBS (pH 7.4) three times, 5 min each, and incubated in secondary antibodies (1:400) for 1 h at 23-25° ° C. Sections were also stained with 4′,6-diamidino-2-phenylindole (DAPI; 1:10,000; Molecular Probes). All images were obtained using a Zeiss AXIO Imager Z2 microscope (Oberkochen, Germany).

Statistical analysis: All quantitative data were collected from experiments performed at least three independent times. The results of three technical or biological replicates are shown and expressed as mean +SD. Differences between groups were assayed using two-tailed student t-test using Microsoft Excel. Significant differences were considered when P<0.05. Data normalization, if applicable, is described in the relevant figure legend.

### Example 1: Derivation of EZH2 ASOs That Reactivate Epigenetically Silenced FMR1 in FXS Neurons

Recently, antisense oligonucleotide (ASO)-based approaches have been developed as effective treatment options for certain central nervous system (CNS) disorders. EZH2 ASOs were derived that could reactivate FMR1 for use as potential FXS therapeutics. Conserved regions between the human and mouse EZH2 genes were identified by bioinformatic analysis, and a series of 10 locked nucleic acid (LNA) ASOs targeting these conserved regions were designed using the LNCASO web server. The LNA ASOs were tested for efficacy in 293T cells by monitoring expression of EZH2 by qRT-PCR. Derivatives of the most efficacious EZH2 ASO were re-synthesized with 2′-O-methoxyethyl-RNA (MOE) modification and have the following sequences: EZH2 ASO-1, &G*&T&C&T&A*C*A*T*G*T*T*T*T*&G&G&T&C*&C, and EZH2 ASO-2, &T*&G&T&C&T*A*C*A*T*G*T*T*T*T*&G&G&T&C*&C (where “&” represents MOE modification, and “*” represents phosphorothioate linkage; GTCTACATGTTTTGGTCC (SEQ ID NO: 1)), and EZH2 ASO-2, &T*&G&T&C&T*A*C*A*T*G*T*T*T*T*&G&G&T&C*&C (TGTCTACATGTTTTGGTCC; SEQ ID NO: 2). A non-targeting control ASO was also synthesized with the sequence &C*&C*&T*&A*&T*A*G*G*A*C*T*A*T*C*C*&A*&G*&G*&A*&A (CCTATAGGACTATCCAGGAA; SEQ ID NO: 30). ASOs were synthesized as MOE gapmers (i.e., 5 MOE nucleotides, 8-10 DNA nucleotides, 5 MOE nucleotides) using standard phosphoramidite methods on a Dr. Oligo 48 synthesizer (Biolytic). Phosphoramidites and other standard reagents were purchased from ChemGenes. Coupling times for MOE nucleotides were extended to 2 minutes. Oligonucleotides were cleaved and deprotected in concentrated aqueous ammonia at 55 °C for 16 hours. ASOs were characterized by liquid chromatography-mass spectrometry (LC-MS) analysis using an Agilent quadruple time of flight (Q-TOF) LC-MS instrument and were desalted using Amicon ultrafiltration columns (3-kDa cutoff).

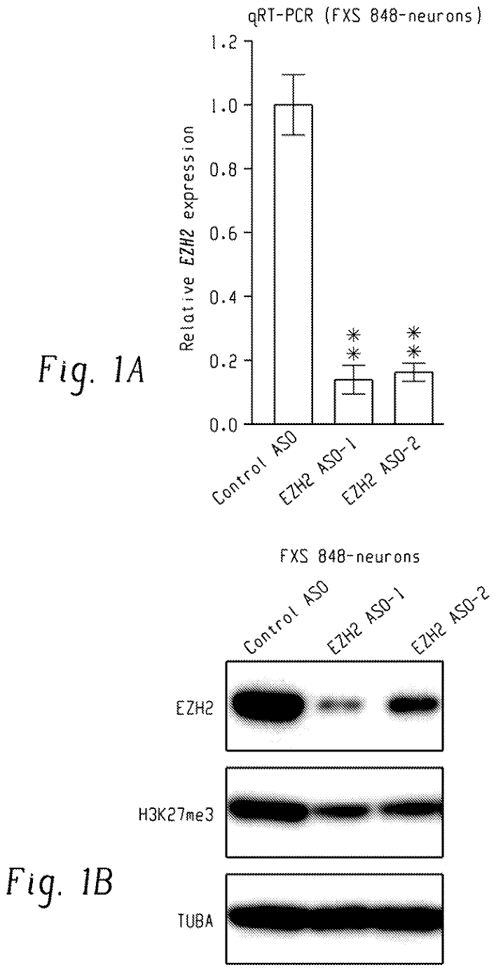

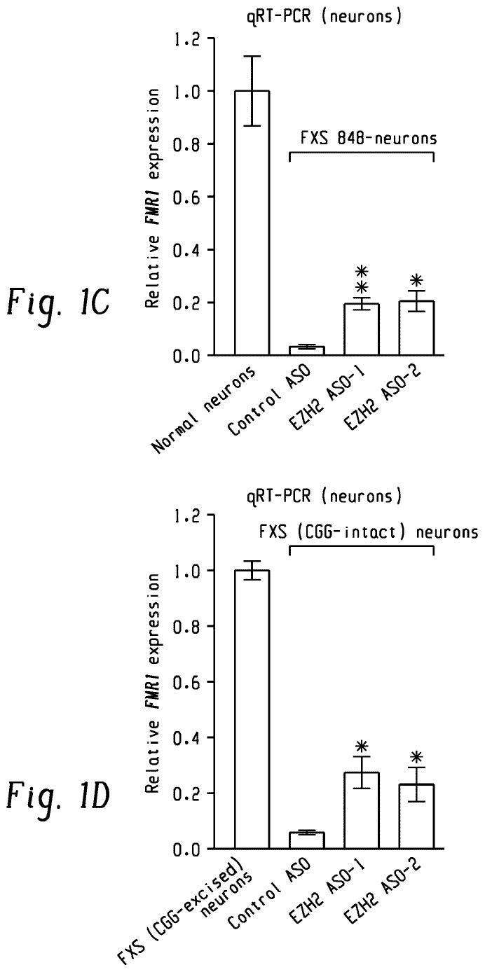

FIG. 1A shows that the two EZH2 ASOs efficaciously reduced EZH2 expression in FXS 848-neurons, a post-mitotic neuronal line generated by transduction of an induced pluripotent stem cell (iPSC) line, derived from a male patient with FXS (FXS 848-iPS3cells, hereafter called FXS 848-iPSCs), with a lentivirus expressing Neurogenin-1 and Neurogenin-2 according to published methods. As expected, the two EZH2 ASOs decreased H3K27me3 levels (FIG. 1B). Treatment of cultured FXS 848-neurons with either of the EZH2 ASOs reactivated FMR1 to approximately 20% of normal levels (FIG. 1C). To confirm this finding, an isogenic pair of FXS cell lines in which the CGG repeats are either intact (CGG-intact) or have been excised using CRISPR/Cas9-mediated deletion (CGG-excised) was used. Consistent with results in FXS 848-neurons, treatment of FXS (CGG- intact) iPSCs and neurons with an EZH2 ASO restored FMR1 mRNA or FMRP protein to Approximately 10-20% of the levels observed in FXS (CGG-excised) neurons (FIG. 1D).

### Example 2: EZH2 ASOs Normalize Characteristic Molecular Abnormalities of FXS Neurons

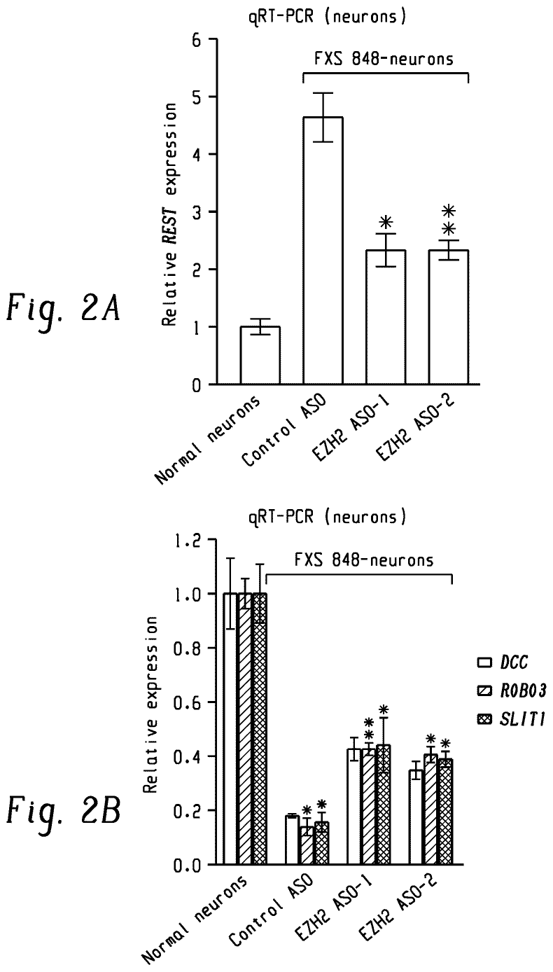

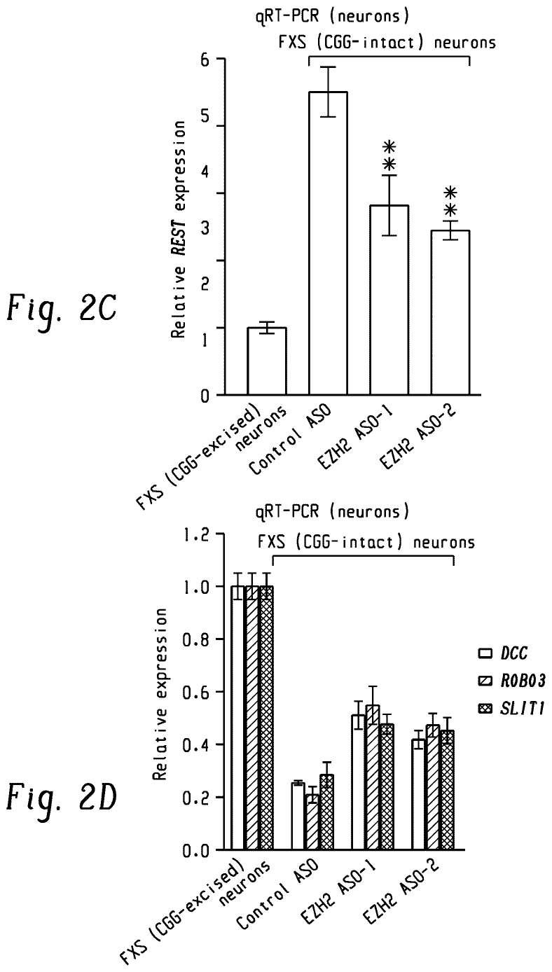

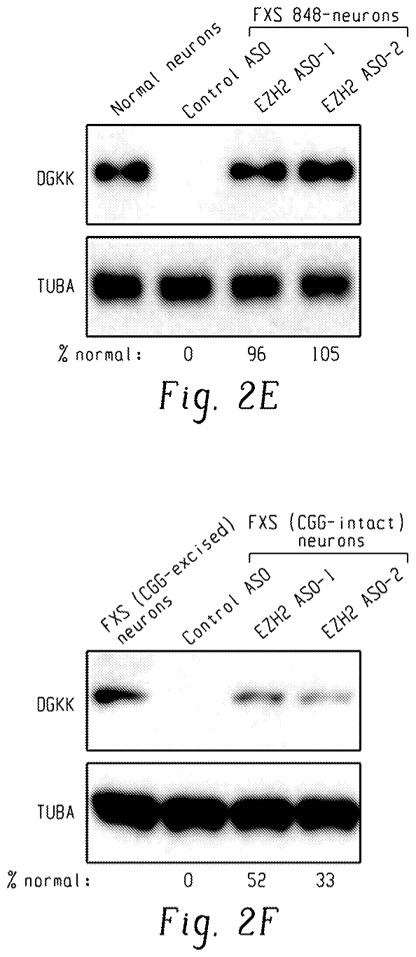

A series of experiments were performed to determine whether the level of FMR1 reactivation was sufficient to normalize characteristic molecular abnormalities of FXS neurons. The transcription factor RE1-Silencing Transcription factor (REST) is a master negative regulator of neurogenesis that controls the pool size and timing of differentiation of various neural lineages. REST is expressed in embryonic stem cells, NPCs, and non- neuronal cells, where it suppresses neuron-specific genes, and is not expressed in differentiated neurons. FMRP helps maintain the levels of a neural-specific microRNA or miRNA (hsa-mir-382), which is a repressor of REST translation. Thus, in the absence of FMRP, the levels of hsa-mir-382 are decreased, preventing the differentiation-dependent downregulation of REST. The resulting higher levels of REST in FXS neurons lead to the suppression of axonal guidance and other genes important for neural development. The EZH2 ASOs substantially decreased expression of REST (FIG. 2A) and increased expression of REST target axonal guidance genes deleted in colorectal carcinoma or netrin receptor DCC (DCC), roundabout homolog 3 (ROB03) and slit homolog 1 protein (SLIT1) (FIG. 2B). Similar results were obtained in neurons derived from the isogenic FXS (CGG-excised/intact) iPSC lines (FIG. 2C and FIG. 2D).

FMRP has been shown to physically associate with the mRNA encoding DGKK, a kinase that controls the switch between diacylglycerol and phosphatidic acid signaling pathways. The absence of FMRP in FXS neurons results in decreased levels of DGKK, which is sufficient to cause dendritic spine abnormalities, synaptic plasticity alterations, and behavioral disorders similar to those observed in Fmr1 knockout mice. Notably, ectopic expression of DGKK rescues the dendritic spine defects of Fmr1 knockout neurons and adeno-associated viral vector delivery of DGKK corrects abnormal diacylglycerol and phosphatidic acid homeostasis and behavioral disorders in Fmr1 knockout mice. Consistent with these published results, immunoblot analysis showed that DGKK was readily detectable in normal but not in FXS 848-neurons (FIG. 2E). Treatment of FXS 848-neurons with EZH2 ASOs increased DGKK levels. Similar results were obtained in neurons derived from the isogenic FXS (CGG-excised/intact) iPSC lines (FIG. 2F).

### Example 3: EZH2 ASOs Correct Electrophysiological Abnormalities in Cultured FXS Neurons

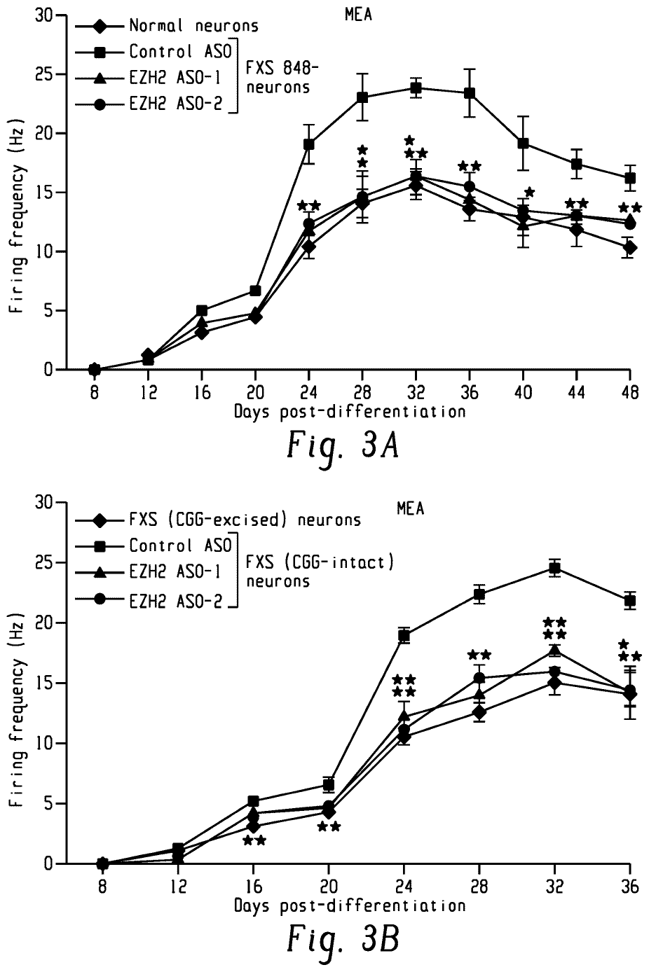

Previous studies have shown that loss of FMRP results in the characteristic electrophysiological abnormality of neuronal hyperexcitability. Reactivation of FMR1 by targeted demethylation of the CGG repeats has been shown to ameliorate the hyperexcitability of FXS neurons. It was thus determined whether FMR1 reactivation resulting from EZH2 ASO treatment could normalize the hyperexcitability of FXS neurons. In these experiments, neuronal hyperexcitability was measured using multielectrode arrays (MEAs). In brief, FXS 848-neurons were treated with an EZH2 ASO and cultured on MEAs, and the spontaneous firing frequency, a measure of excitability, was monitored over a 48-day time course. Consistent with previous studies, the MEA results revealed that compared to their normal counterparts, FXS 848-neurons displayed increased firing frequency, indicative of hyperexcitability (FIG. 3A). Treatment of FXS 848-neurons with an EZH2 ASO corrected the hyperexcitability of FXS 848-neurons. Similar results were obtained in neurons derived from the isogenic FXS (CGG-excised/intact) iPSC lines (FIG. 3B). Thus, the level of FMR1 reactivation obtained by EZH2 ASOs is sufficient to correct the hyperexcitability of FXS neurons.

### Example 4: EZH2 ASOs Reactivate FMR1 Expression in Human FXS NPCS Engrafted Within the Brains of Mice

Currently, there is no FXS mouse model to directly analyze the therapeutic benefit of FMR1 reactivation, because incorporation of the CGG repeat expansion in mice fails to recapitulate the DNA hypermethylation and transcriptional silencing of FMR1 that occurs in human cells. As an alternative approach, two groups have reported the development of a mouse engraftment model to analyze FMR1 reactivation in the central nervous system (CNS). In this model, FXS NPCs are stereotaxically injected into the brains of mice, and the grafts are subsequently analyzed for human FMR1/FMRP expression.

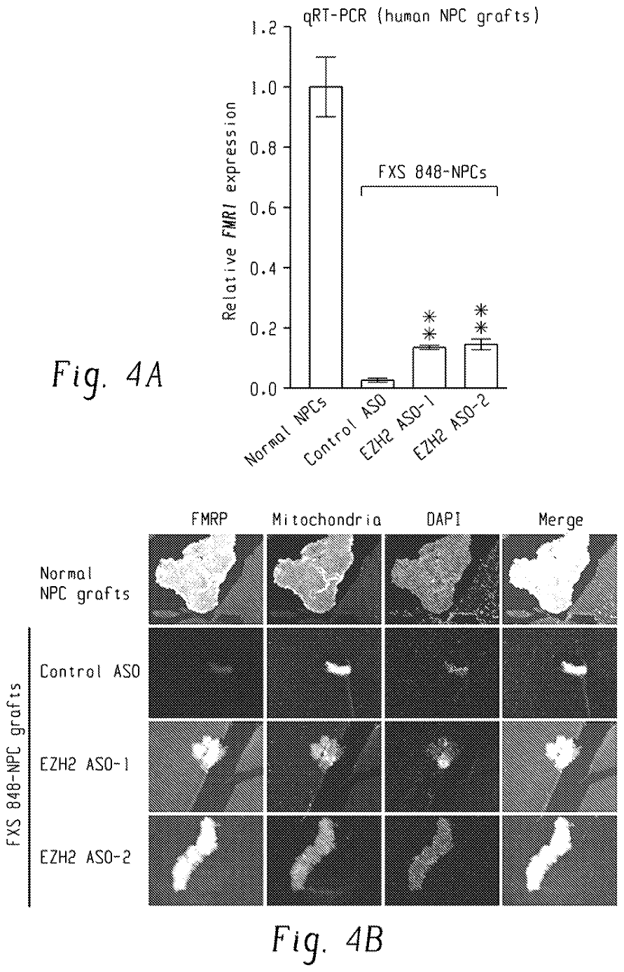

To test whether EZH2 ASOs could reactivate epigenetically silenced FMR1 in this mouse engraftment model, FXS 848-NPCs were stereotaxically injected into the cerebral lateral ventricles of mice. Three days after transplantation, mice received an intracerebroventricular injection of an EZH2 ASO, and seven days later, mice were sacrificed, and the brain grafts collected and dissected. Analysis by qRT-PCR using primers specific for the human FMR1 transcript showed that EZH2 ASOs reactivated expression of FMR1 to ~15% of normal levels (FIG. 4A). Furthermore, immunofluorescence staining of mouse brain sections with a human-specific anti-FMRP antibody revealed that EZH2 ASO treatment also resulted in increased FMRP levels (FIG. 4B). Collectively, these results demonstrate that EZH2 ASOs can reactivate FMR1 in vivo.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. An EZH2 antisense oligonucleotide epigenetic modulator of FMR1 having a length of 16-25 nucleotides and which comprises a sequence at least 90% identical to GTCTACATGTTTTGGTCC (SEQ ID NO: 1) or TGTCTACATGTTTTGGTCC (SEQ ID NO: 2), wherein one or more T nucleotides is optionally replaced with U, and one or more C nucleotides is optionally replaced with 5-methyl C.

2. The EZH2 antisense oligonucleotide epigenetic modulator of FMR1 of claim 1, comprising a modification of a ribose group, wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH2, a constrained nucleotide, a tricyclo-DNA modification, a morpholino nucleotide, or a combination thereof.

3. The EZH2 antisense oligonucleotide epigenetic modulator of FMR1 of claim 1, comprising a modification of a phosphate group, wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof.

4. The EZH2 antisense oligonucleotide epigenetic modulator of FMR1 of claim 1, wherein SEQ ID NO: 1 comprises &G*&T&C&T&A*C*A*T*G*T*T*T*T*&G&G&T&C*&C, or SEQ ID NO: 2 comprises &T*&G&T&C&T*A*C*A*T*G*T*T*T*T*&G&G&T&C*&C, wherein & is a 2′-deoxy, 2′-O-(2-methoxyethyl) modification and * is a phosphorothioate linkage.

5. A pharmaceutical composition comprising the EZH2 antisense oligonucleotide epigenetic modulator of FMR1 of claim 1 and a pharmaceutically acceptable carrier.

6. A method of treating a fragile X-associated disorder in a patient in need of such treatment, the method comprising administering to a subject in need thereof the EZH2 antisense oligonucleotide epigenetic modulator of FMR1 of claim 1 or a pharmaceutical composition comprising the EZH2 antisense oligonucleotide epigenetic modulator of FMR1 and a pharmaceutically acceptable carrier.

7. The method of claim 6, wherein the EZH2 antisense oligonucleotide epigenetic modulator of FMR1 or the pharmaceutical composition is administered intravenously, intra-arterially, intrathecally, intraventricularly, intramuscularly, intradermally, subcutaneously, intracranially, or spinally.

8. The method of claim 5, wherein the fragile X-associated disorder is fragile X syndrome (FXS), fragile X-associated primary ovarian insufficiency (FXPOI), or fragile X-associated tremor/ataxia syndrome (FXTAS).

9. The pharmaceutical composition of claim 5, further comprising an antisense oligonucleotide that decreases splicing between Exons 1 and 2 of FMR1 -217 which is complementary to a sequence that is at least 90% identical to 18-29 contiguous nucleotides of SEQ ID NO: 5
(SEQ ID NO: 5)
AGCGGGGGGCGCTTCAGCCGGGCCGCCTCCTGCAGCGCCAAGAGGGCTTC
AGGTCTCCTTTGGCTTCTCTTTTCCGGTCTAGCATTGGGACTTCGGAGAG
CTCCACTGTTCTGGGCGAGGGCTGTGAAGAAAGAGTAGTAAGAAGCGGTA
GTCGGCACCAAATCACAATGGCAACTGATTTTTAGTGGCTTCTCTTTGTG
GATTTCGGAGGAGATTTTAGATCCAAAAGTTTCAGGAAGACCCTAACATG
GCCCAGCAGTGCATTGAAGAAGTTGATCATCGTGAATATTCGCGTCCCCC
TTTTTGTTAAACGGGGTAAATTCAGGAATGCACATGCTTCAGCGTCTAAA
ACCATTAGCAGCGCTGCTACTTAAAAATTGTGTGTGTGTGTTTAAGTTTC
CAAAGACCTAAATATATGCC.

10. The pharmaceutical composition of claim 9, wherein the ASO that decreases splicing between Exons 1 and 2 of FMR1 -217 is at least 90% identical to:
(W-704)
(SEQ ID NO: 6)
AGAAGCCAAAGGAGACCTGA,
(W-705)
(SEQ ID NO: 7)
AAAGAGAAGCCAAAGGAGAC,
(W-706)
(SEQ ID NO: 8)
CTAGACCGGAAAAGAGAAGCCA,
(W-707)
(SEQ ID NO: 9)
ATGCTAGACCGGAAAAGAGAA,
(W-708)
(SEQ ID NO: 10)
CAATGCTAGACCGGAAAAGA,
(W-709)
(SEQ ID NO: 11)
AAGTCCCAATGCTAGACCGGA
(W-710)
(SEQ ID NO: 12)
TCTCCGAAGTCCCAATGCTA,
(W-711)
(SEQ ID NO: 13)
GAGCTCTCCGAAGTCCCA,
(W-712)
(SEQ ID NO: 14)
AGAACAGTGGAGCTCTCCGA,
(W-713)
(SEQ ID NO: 15)
CGCCCAGAACAGTGGAGCTC,
(W-714)
(SEQ ID NO: 16)
CCTCGCCCAGAACAGTGGAG,
(2831)
(SEQ ID NO: 17)
CAGTGGAGCTCTCCGAAGTCC,
(2832)
(SEQ ID NO: 18)
CCCAGAACAGTGGAGCTCTCC;
(2833)
(SEQ ID NO: 19)
CACAGCCCTCGCCCAGAACA,
(SEQ ID NO: 20)
TTCTTCACAGCCCTCGCCCA,
(SEQ ID NO: 21)
TCTTTCTTCACAGCCCTCGCCCAGAACAGTGGAGCTCTCCGAAGTCCCAA
TGCTAGACCGGAAAAGAGAAGCCAAAGGAGACCTGA,
(SEQ ID NO: 22)
TCTCCGAAGTCCCAATGCTAGACCGGAAAAGAGAAGCCAAAGGAGACCTG
A,
(SEQ ID NO: 23)
TCTTTCTTCACAGCCCTCGCCCAGAACAGTGGAGCTCTCCGAAGTCCCAA
TG,
or
(SEQ ID NO: 24)
TTCTTCACAGCCCTCGCCCAGAACAGTGGAGCTCTCCGAAGTCCCA,
wherein one or more T nucleotides is optionally replaced with U, and one or more C nucleotides is optionally replaced with 5-methyl C.

11. The pharmaceutical composition of claim 9, wherein the antisense oligonucleotide that decreases splicing between Exons 1 and 2 of FMR1-217 comprises a modification of a ribose group, wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH₂, a constrained nucleotide, a tricyclo-DNA modification, a morpholino nucleotide, or a combination thereof.

12. The pharmaceutical composition of claim 9, wherein the antisense oligonucleotide that decreases splicing between Exons 1 and 2 of FMR1-217 comprises a modification of a phosphate group and wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof.

13. A method of treating a fragile X-associated disorder in a patient in need of such treatment, comprising administering to a subject in need thereof the pharmaceutical composition of claim 9.

14. A method of treating a fragile X-associated disorder in a patient in need of such treatment, comprising
administering to the subject an EZH2 antisense oligonucleotide epigenetic modulator of FMR1 having a length of 16-25 nucleotides, and
simultaneously or sequentially administering to the subject an antisense oligonucleotide that decreases splicing between Exons 1 and 2 of FMR1-217 which is complementary to a sequence that is at least 90% identical to 18-29 contiguous nucleotides of SEQ ID NO: 5.

15. The method of claim 14, wherein the EZH2 antisense oligonucleotide epigenetic modulator of FMR1 has a length of 16-25 nucleotides and comprises a sequence at least 90% identical to GTCTACATGTTTTGGTCC (SEQ ID NO: 1) or TGTCTACATGTTTTGGTCC (SEQ ID NO: 2), wherein one or more T nucleotides is optionally replaced with U, and one or more C nucleotides is optionally replaced with 5-methyl C.

16. The method of claim 14, wherein at least one of:
the EZH2 antisense oligonucleotide epigenetic modulator of FMR1 comprises a modification of a ribose group, wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH2, a constrained nucleotide, a tricyclo-DNA modification, a morpholino nucleotide, or a combination thereof;
the EZH2 antisense oligonucleotide epigenetic modulator of FMR1 comprises a modification of a phosphate group, wherein the modification of the phosphate group comprises a phosphorothioate, a phosphoramidate, a phosphorodiamidate, a phosphorodithioate, a phosphonoacetate (PACE), a thiophosphonoacetate (thioPACE), an amide, a triazole, a phosphonate, a phosphotriester, or a combination thereof; or a combination thereof.

17. (canceled)

18. The method of claim 15, wherein the EZH2 antisense oligonucleotide epigenetic modulator of FMR1 of SEQ ID NO: 1 comprises &G*&T&C&T&A*C*A*T*G*T*T*T*T*&G&G&T&C*&C, or SEQ ID NO: 2 comprises &T*&G&T&C&T*A*C*A*T*G*T*T*T*T*&G&G&T&C*&C, wherein & is a 2′-deoxy, 2′-O-(2-methoxyethyl) modification and * is a phosphorothioate linkage.

19. The method of claim 14, wherein the antisense oligonucleotide that decreases splicing between Exons 1 and 2 of FMR1-217 is at least 90% identical to:
(W-704)
(SEQ ID NO: 6)
AGAAGCCAAAGGAGACCTGA,
(W-705)
(SEQ ID NO: 7)
AAAGAGAAGCCAAAGGAGAC,
(W-706)
(SEQ ID NO: 8)
CTAGACCGGAAAAGAGAAGCCA,
(W-707)
(SEQ ID NO: 9)
ATGCTAGACCGGAAAAGAGAA,
(W-708)
(SEQ ID NO: 10)
CAATGCTAGACCGGAAAAGA,
(W-709)
(SEQ ID NO: 11)
AAGTCCCAATGCTAGACCGGA
(W-710)
(SEQ ID NO: 12)
TCTCCGAAGTCCCAATGCTA,
(W-711)
(SEQ ID NO: 13)
GAGCTCTCCGAAGTCCCA,
(W-712)
(SEQ ID NO: 14)
AGAACAGTGGAGCTCTCCGA,
(W-713)
(SEQ ID NO: 15)
CGCCCAGAACAGTGGAGCTC,
(W-714)
(SEQ ID NO: 16)
CCTCGCCCAGAACAGTGGAG,
(2831)
(SEQ ID NO: 17)
CAGTGGAGCTCTCCGAAGTCC,
(2832)
(SEQ ID NO: 18)
CCCAGAACAGTGGAGCTCTCC;
(2833)
(SEQ ID NO: 19)
CACAGCCCTCGCCCAGAACA,
(SEQ ID NO: 20)
TTCTTCACAGCCCTCGCCCA,
(SEQ ID NO: 21)
TCTTTCTTCACAGCCCTCGCCCAGAACAGTGGAGCTCTCCGAAGTCCCAA
TGCTAGACCGGAAAAGAGAAGCCAAAGGAGACCTGA,
(SEQ ID NO: 22)
TCTCCGAAGTCCCAATGCTAGACCGGAAAAGAGAAGCCAAAGGAGACCTG
A,
(SEQ ID NO: 23)
TCTTTCTTCACAGCCCTCGCCCAGAACAGTGGAGCTCTCCGAAGTCCCAA
TG,
or
(SEQ ID NO: 24)
TTCTTCACAGCCCTCGCCCAGAACAGTGGAGCTCTCCGAAGTCCCA,
wherein one or more T nucleotides is optionally replaced with U, and one or more C nucleotides is optionally replaced with 5-methyl C.

20. The method of claim 14, wherein at least one of:
the antisense oligonucleotide that decreases splicing between Exons 1 and 2 of FMR1-217 comprises a modification of a ribose group, wherein the modification of the ribose group comprises 2′-O-methyl, 2′-fluoro, 2′-deoxy, 2′-O-(2-methoxyethyl) (MOE), 2′-O-alkyl, 2′-O-alkoxy, 2′-O-alkylamino, 2′-NH₂, a constrained nucleotide, a tricyclo-DNA modification, a morpholino nucleotide, or a combination thereof;
the antisense oligonucleotide that decreases splicing between Exons 1 and 2 of FMR1-217 comprises a modification of a phosphate group, wherein the modification of the phosphate group comprises a phosphorothioate, a phosphoramidate, a phosphorodiamidate, a phosphorodithioate. a phosphonoacetate (PACE), a thiophosphonoacetate (thioPACE), an amide, a triazole, a phosphonate, a phosphotriester, or a combination thereof; or a combination thereof.

21. (canceled)

22. The method of claim 14, wherein the fragile X-associated disorder is fragile X syndrome (FXS), fragile X-associated primary ovarian insufficiency (FXPOI), or fragile X-associated tremor/ataxia syndrome (FXTAS).
